(19) United States
(12) Patent Application Publication
(10) Pub. No.: US 2011/0263623 A1
(43) Pub. Date: Oct. 27, 2011

(54) PROCESS FOR PREPARATION OF BOSENTAN

(75) Inventors: Kaushilk Sata, Ahmedabad (IN); Bipin Pandey, Ahmedabad (IN)

(73) Assignee: CADILA HEALTHCARE LIMITED, Ahmedabad, Gujarat (IN)

(21) Appl. No.: 13/058,285

(22) PCT Filed: Aug. 7, 2009
(86) PCT No.: PCT/IN09/00443

§ 371 Date: May 6, 2011

(30) Foreign Application Priority Data
Aug. 12, 2008 (IN) 1705/MUM/2008
Mar. 12, 2009 (IN) 537/MUM/2009

(51) Int. Cl. A61K 31/513; A61P 9/12; C07D 403/04

(52) U.S. Cl. 514/269; 544/296

(57) ABSTRACT

The present invention provides improved processes for preparing Bosentan. The present invention provides novel intermediates like 4,6-dihydroxy-5-(2-methoxy phenoxy)[2,2′]bipyrimidine of formula (II) and N-(6-Chloro-5-(2-ethoxyphenoxy)[2,2′-bipyrimidinyl]-4-t-butyl benzenesulfonamide cesium salt and process for preparation thereof. The invention also disclosed novel polymorphic form of the intermediates.

### FIELD OF INVENTION

The present invention provides an improved process for preparation of Bosentan. The present invention also provide a novel intermediate of Bosentan. The process of the present invention uses a novel intermediate, and also provides for its polymorphic forms.

Priority is claimed to provisional application No. 1705/MUM/2008, filed on Aug. 12, 2008 and provisional application No. 537/MUM/2009, filed on Mar. 12, 2009.

### BACKGROUND OF THE INVENTION

Bosentan is a dual endothelin receptor antagonist important in the treatment of pulmonary artery hypertension (PAH). Bosentan is marketed under the trade name Tracleer® by Actelion Pharmaceutical. Bosentan is chemically known as 4-tert-butyl-N-[6-(2-hydroxyethoxy)-5-(2-methoxyphenoxy)-2-(2-pyrimidinyl)pyrimidin-4-yl]benzenesulfonamide has the structural formula as shown in formula (I).

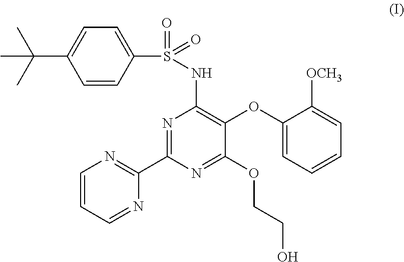

U.S. Pat. No. 5,292,740 disclose several molecules, which are sulfonamide derivatives and includes Bosentan. In the specification, various synthetic schemes for preparation of the molecules and for their intermediates are disclosed. The preparation method involves coupling of N-[6-chloro-5(2-methoxyphenoxy)-2-(2-pyrimidinyl)-pyrimidin-4-yl]-4-tert-butyl-benzenesulfonamide and sodium ethylene glycolate in ethylene glycol at 100° C. (Scheme 1).

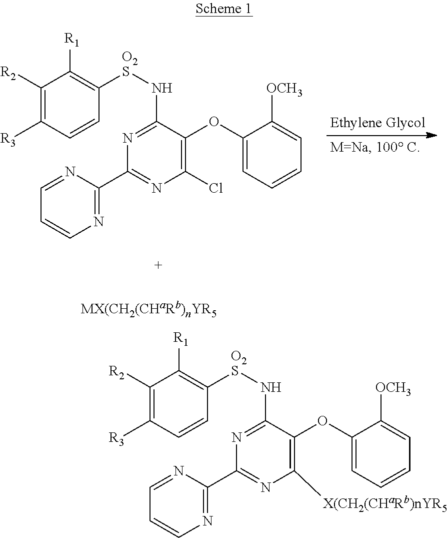

WO 200155120 discloses a process for the preparation of ethylene glycol sulfonamide derivatives. The process involves the step of conversion of pyrimidinedion to pyrimidine dihalide using suitable dehydrohalogenating agent. In the second step the pyrimidine dihalide is reacted with a sulfonamide in presence of suitable base and suitable phase transfer catalyst to obtain the pyrimidine monohalide derivative. Third step involves the reaction of pyrimidine monohalide derivative with mono-protected ethylene glycol in nonpolar aprotic solvents in the presence of a base to obtain mono-protected ethylene glycol sulfonamide derivatives, which after removing the protecting group produces the ethylene glycol sulfonamide derivatives (Scheme 2). This specification also discloses the preparation of Bosentan according to the above process.

However WO 200155120 also states, the disadvantages of the process disclosed in U.S. Pat. No. 5,292,740. It states that the disadvantages of using a monoanion of ethylene glycol is the formation of undesired ethylene glycol bissulfonamide, in which two molecules of the pyrimidine monohalide are coupled with one molecule of ethylene glycol. The removal of bis-sulfonamide compound requires costly and laborious separation steps to obtain a pharmaceutically suitable pure ethylene glycol sulfonamide compound. Another drawback of this process is the need for isolating a pyrimidine dihalide, which is believed to be a potent sensitiser.

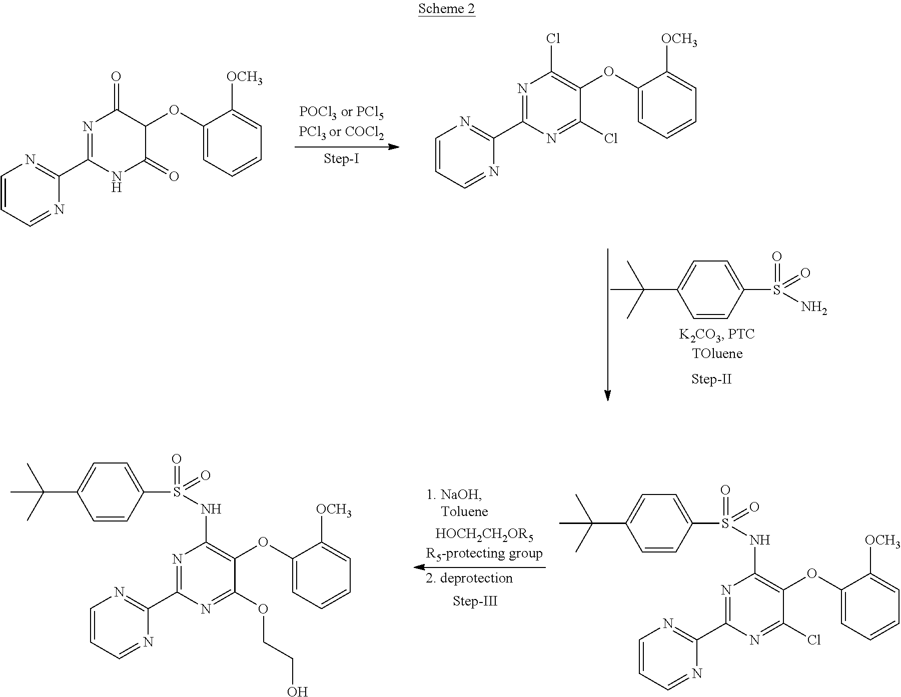

The main disadvantages of the process of Scheme 2 is that, using mono-protected ethylene glycol sulfonamide requires additional deprotection step, so the number of reaction step is increased. Also, the process requires the use of a phase transfer catalyst, which increases the cost and operational ease.

WO2008135795 discloses novel polymorphic forms of Bosentan and processes for their preparation. They have disclosed crystalline form I (M.P.-148° C.), crystalline form II (M.P.-144° C.), crystalline form III (endothermic peak-174° C. and endothermic peak-246° C.), crystalline form IV (M.P.-210° C.) and an amorphous form of Bosentan. WO2009053748 discloses novel polymorphic forms V to VIII of Bosentan and processes for their preparation.

WO2009047637 discloses novel polymorphic forms A1, A2 and A4 of Bosentan and processes for their preparation.

WO2009004374 discloses a process for the preparation of Bosentan comprising, N-(6-chloro-5-(2-methoxyphenoxy)[2,2′-bipyrimidinyl]-4-t-butyl-benzene sulfonamide reacted with ethanediol in presence of hydroxide ions. The document only describes the use of one sodium hydroxide. No other alkali metal hydroxide ions are used and no other examples are provided in the application.

Thus, there remains a need for developing a process, which overcomes one or more of the deficiencies of the prior art and thereby develop an improved process for preparing Bosentan. We herein disclose an improved process for preparing Bosentan wherein in situ monoanion of ethylene glycol is used and surprisingly, found that the formation of bis-impurity compound is substantially reduced. This helps in the conversion to pure final product. Here, the amount of the ethylene glycol used is very less in comparison with ethylene glycol used in method disclosed in U.S. Pat. No. 5,292,740. This route also avoids the use of mono-protected ethylene glycol. We herein also disclose a new impurity of Bosentan, process of its preparation, which may be used as a reference standard.

### BRIEF DESCRIPTION OF THE DRAWINGS

FIG. 1 is a powder X-ray diffraction (XRPD) pattern of the crystalline 4,6-dihydroxy-5-(2-methoxy phenoxy)[2,2′]bipyrimidine according to the present invention.

FIG. 2 is a powder X-ray diffraction (XRPD) pattern of the crystalline 4,6-dihydroxy-5-(2-methoxy phenoxy)[2,2′]bipyrimidine according to the present invention.

FIG. 3 is a powder X-ray diffraction (XRPD) pattern of the crystalline Bosentan obtained according to the present invention.

FIG. 4 is a powder X-ray diffraction (XRPD) pattern of the crystalline cesium salt of N-(6-Chloro-5-(2-methoxyphenoxy)[2,2′-bipyrimidinyl]-4-t-butyl benzenesulfonamide obtained according to the present invention.

FIG. 5 is a differential scanning calorimetric curve of the Crystalline Bosentan obtained according to the present invention.

### OBJECTS OF THE INVENTION

The object of the present invention is to provide improved processes for preparing Bosentan.

In an embodiment a novel intermediate of formula (II) is used for the preparation of Bosentan.

In an embodiment is provided a process for preparing Bosentan using ethanediol.

In an embodiment is provided a novel intermediate of formula (II) and process for preparation thereof.

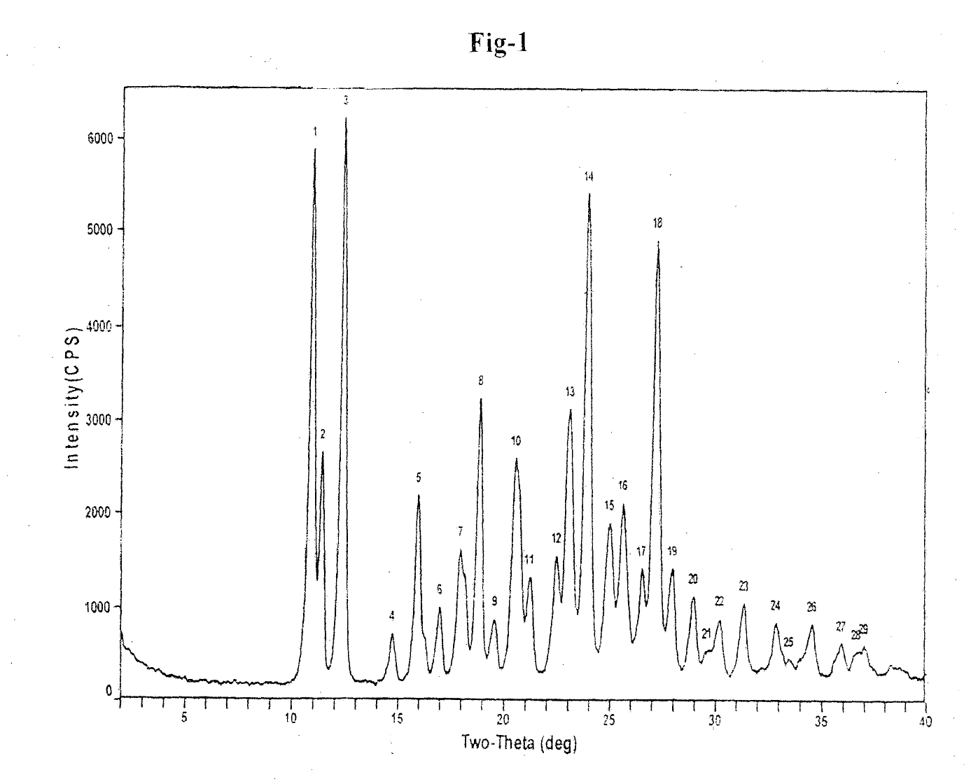

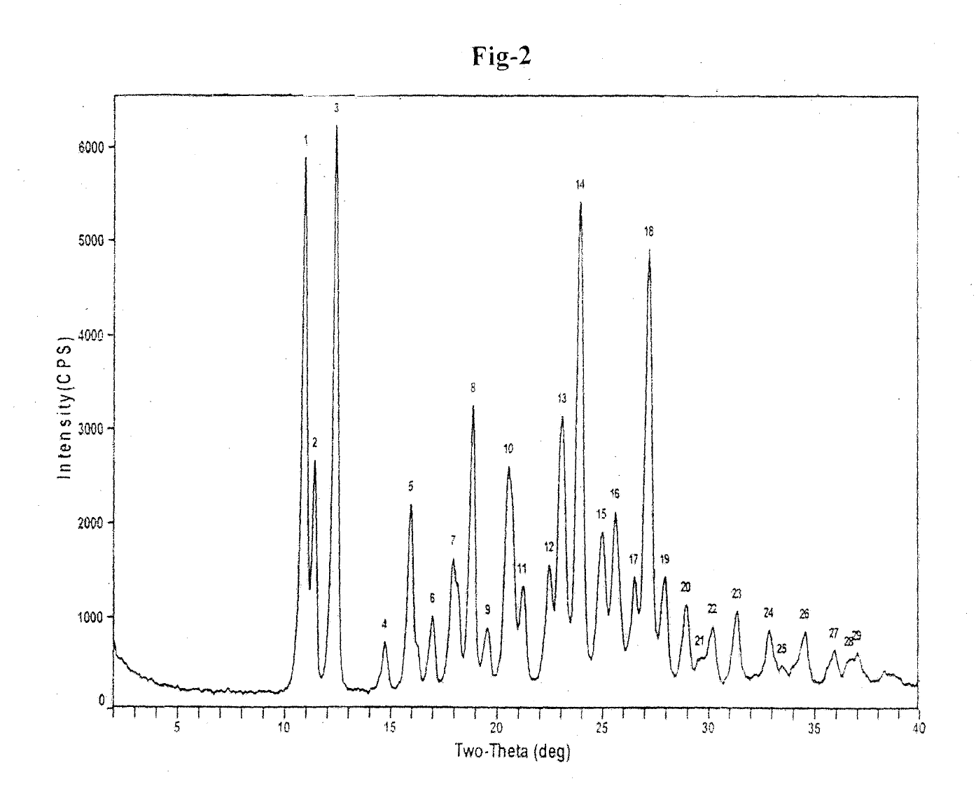

In one of the embodiment is provided two novel polymorphic forms of 4,6-dihydroxy-5-(2-methoxy phenoxy)[2,2′]bipyrimidine of formula (II), characterized by powder X-ray diffraction (XRPD) pattern as provided in FIG. 1 and FIG. 2 respectively.

In a still further embodiment is provided a novel cesium salt of N-(6-chloro-5-(2-methoxyphenoxy)[2,2′-bipyrimidinyl]-4-t-butyl benzene sulfonamide.

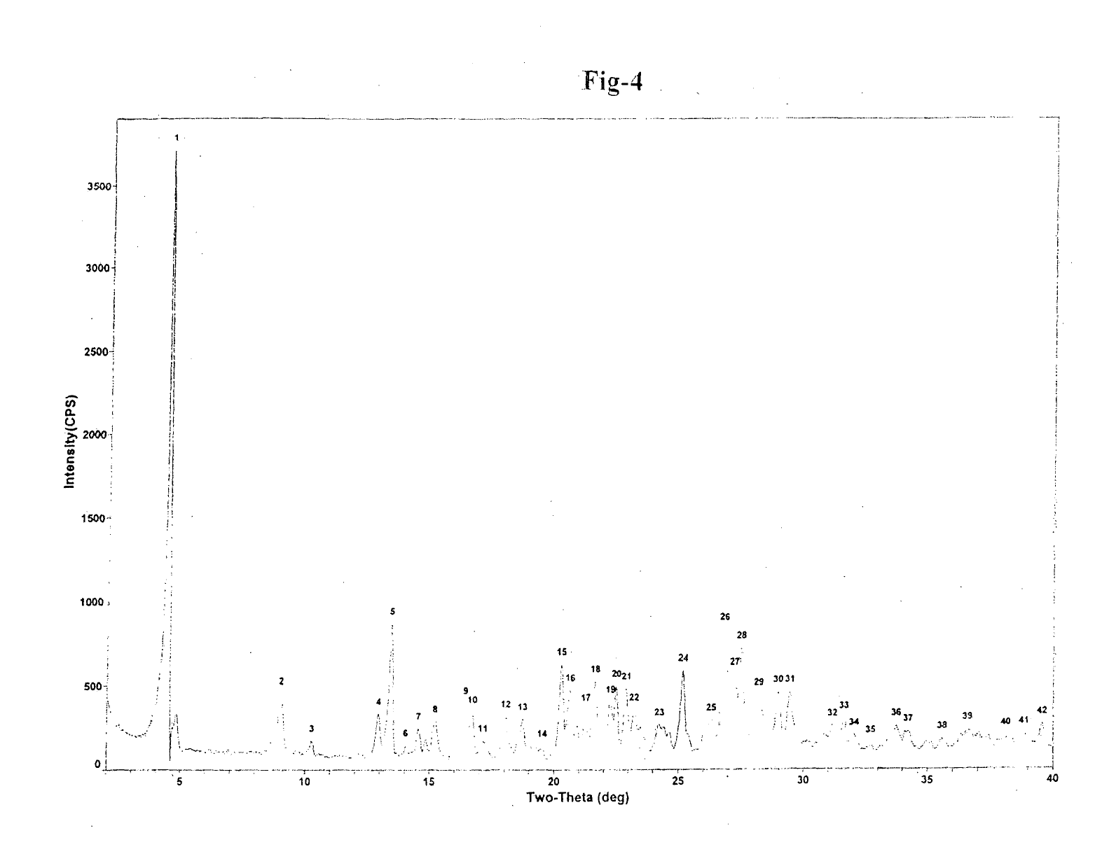

In a still further embodiment a cesium salt of N-(6-chloro-5-(2-methoxyphenoxy)[2,2′-bipyrimidinyl]-4-t-butyl benzene sulfonamide is characterized by powder X-ray diffraction-(XRPD) pattern as provided in FIG. 4.

In yet another embodiment is provided a process for preparing Bosentan using the cesium salt of N-(6-chloro-5-(2-methoxyphenoxy)[2,2′-bipyrimidinyl]-4-t-butyl benzene sulfonamide.

In a still another embodiment is provided a process for introduction of hydroxyethoxy side chain in Bosentan in presence of a suitable base as provided elsewhere in the specification, optionally in presence of a suitable phase transfer catalyst.

In a still further embodiment is provided a novel compound N,N-(5-(2-methoxyphenoxy)-2,2′-bipyrimidine-4,6-diyl)bis(4-tert-butylbenzenesulfonamide of formula (III) and process for preparation thereof.

In yet another embodiment is provided the use of compound of formula (III) as a reference standard for Bosentan.

The above and other embodiments are further described in the following paragraphs.

### DETAILED DESCRIPTION

As used herein, the term “reflux temperature” refers to the boiling point of the solvent.

As used herein, the term “PXRD” refers to powder X-ray diffraction.

As used herein, the term “THF” refers to tetrahydrofuran, the term “DCM” refers to dichloromethane, the term “DMF” refers to dimethyl formamide, the term “DIPE” refers to diisopropyl ether, the term “EG” refers to ethylene glycol, the term “PTC” refers to phase transfer catalyst, the term “DMAP” refers to 4-dimethyl amino pyridine.

The improved process for preparing Bosentan using novel intermediate of formula (II) and N-(6-Chloro-5-(2-methoxyphenoxy)[2,2′-bipyrimidinyl]-4-t-butyl benzenesulfonamide cesium salt, is described in the following scheme:

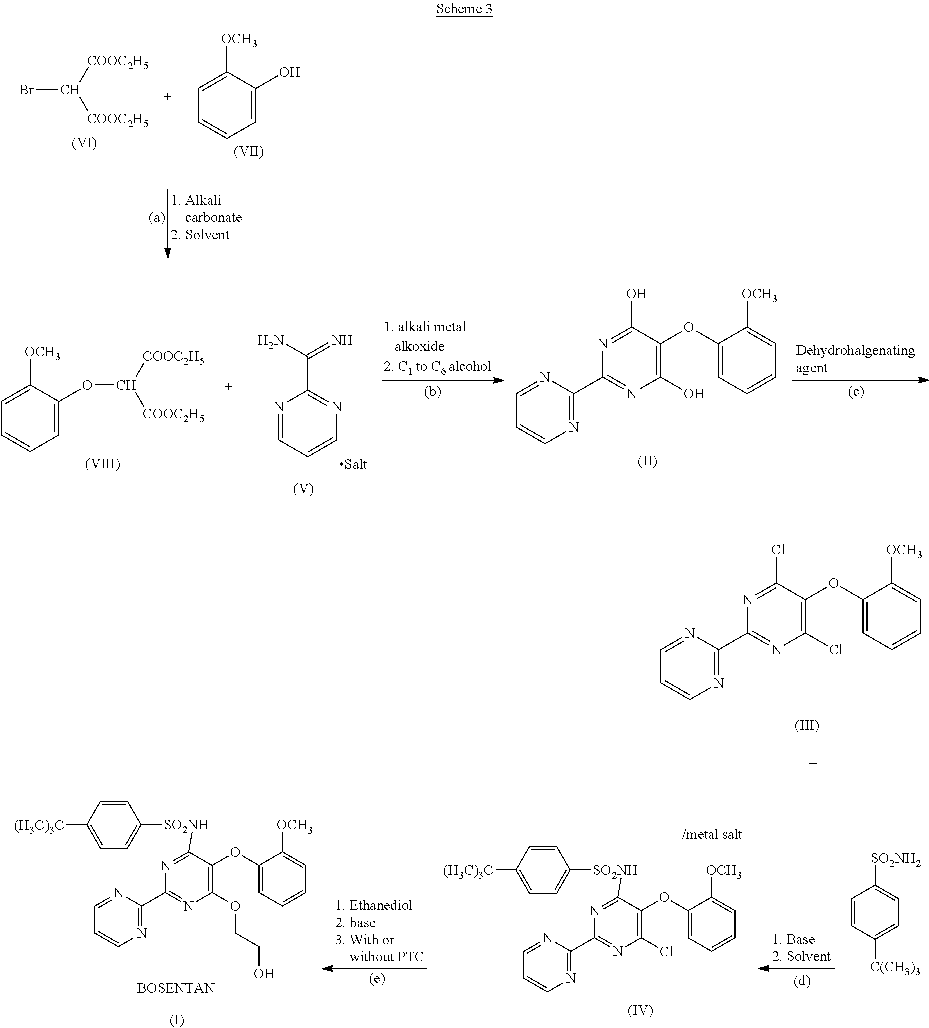

(a) reaction of 2-bromodiethyl malonate (VI) with 2-methoxy-phenol (VII) in presence of suitable alkali carbonate in suitable solvent gives 2-(2-methoxyphenoxy) malonic acid diethyl ester (VIII).

Suitable alkali carbonates used in step (a) may be selected from sodium carbonate, potassium carbonate, lithium carbonate and cesium carbonate, preferably potassium carbonate.

Suitable solvent(s) used in the step (a) may be selected from the solvents like ketones such as acetone; polar solvents such as dimethyl formamide, dimethyl sulfoxide, dimethyl acetamide, acetonitrile, n-butanol, dioxane or their suitable mixtures. The reaction is preferably carried out in acetone.

(b) pyrimidine-2-carboxamidine is converted to its suitable acid addition salts such as hydrochloride, hydrobromide, acetate, sulfate and benzene sulfonate, preferably acetate.

The salt of the pyrimidine-2-carboxamidine (V) is reacted with 2-(2-methoxyphenoxy) malonic acid diethyl ester (VIII) in presence of suitable alkali metal alkoxide to give pyrimidine diol derivative of formula (II), [(4,6-dihydroxy-5-(2-methoxy phenoxy)[2,2′]bipyrimidine], having 97-99% purity. Surprisingly it was found that the compounds of formula (II) prepared using different pyrimidine-2-carboxamidine salt, gives different proportion of water by KF.

Suitable base(s) used in step-(b) may be selected from the alkali metal alkoxides such as sodium ethoxide, sodium methoxide, potassium t-butoxide, sodium t-butoxide and like, preferably sodium methoxide.

Solvent(s) used in step-(b) may be selected from C_{I} to C₆ alcohol such as ethanol, methanol, isopropanol and t-butanol, preferably methanol. The duration of the reaction may vary from 2 to 8 hrs, more specifically 3 to 5 hrs.

In one embodiment, the present invention provides a new intermediate compound of formula (II). In one specific embodiment, the compound of formula (II) contains about 6-10% water by weight. This form of compound (II) on drying in an oven at 100-120° C. for 6-8 hrs surprisingly provides another form of the compound of formula (II) which contain 0.1-0.5% water by weight. Both the compounds have been characterized by PXRD peaks and shows different PXRD peaks pattern. Thus, the present invention provides two polymorphic form of compound of formula (II).

The complete x-ray powder spectrum, was recorded with a Rigaku D/Max 2200 VPC X-ray powder diffractometer model using copper radiation. The X-ray diffraction pattern was recorded by keeping the instrument parameters as below:

X-ray: Cu/40 kv/30 mA, Diverging slit: 1°, Scattering slit: 1°, Receiving slit: 0.15 mm, Monochromator RS: 0.8 mm, Counter: Scintillation counter,

Scan mode: Continuous, Scan speed: 3.000°/min., Sampling width: 0.020°, Scan axes: 2 theta vs CPS, Scan range: 2° to 40.0°, Theta offset: 0.000

The infrared (IR) spectrum has been recorded on a Shimadzu FTIR-8400 model spectrophotometer, between 450 cm⁻¹ and 4000 cm⁻¹, with a resolution of 4 cm⁻¹ in a KBr pellet.

(c) The compound of formula (II) is converted into 4,6-dichloro-5-(2-methoxy phenoxy) [2,2′]bipyrimidine of formula (III) by using suitable dehydrohalogenating agent in presence of a suitable base.

Suitable dehydrohalogenating agents may be selected from phosphorous oxychloride, phosphorous pentachloride, phosphorous trichloride, oxalyl chloride, pyrophosphorous oxychloride and like or their suitable mixtures.

Suitable base(s) used in step (c) may be selected from tertiary amines such as triethyl amine, trimethyl amine, triisopropyl amine and diisopropyl ethylamine, preferably triethyl amine. The duration of the reaction may vary from 2 to 24 hrs, more specifically 15 to 20 hrs.

(d) 4,6-Dichloro-5-(2-methoxy phenoxy) [2,2′]bipyrimidine of formula (III), is reacted with 4-tert-butyl-benzene sulfonamide in presence of suitable base selected from alkali metal carbonates, alkali earth metal carbonates and alkali hydroxides in presence of a suitable solvent to obtain N-(6-Chloro-5-(2-methoxyphenoxy)[2,2′-bipyrimidinyl]-4-t-butyl benzene sulfonamide or its metal salt of formula (IV). Suitable alkali carbonates used may be selected from potassium carbonate, sodium carbonate, cesium carbonate and lithium carbonate; alkali earth metal carbonate may be selected from magnesium carbonate, zinc carbonate, most preferably cesium carbonate; alkali hydroxides used may be selected from sodium hydroxide, potassium hydroxide and lithium hydroxide.

Suitable solvent used in step-(d) may be selected from suitable polar solvents, such as dimethyl formamide, dimethyl sulfoxide, dimethyl acetamide; or non polar solvents such as toluene and cyclohexane or their suitable mixture. The reaction is preferably carried out in dimethyl formamide.

The duration of the reaction may vary from 3 to 30 hrs, more specifically 15 to 18 hrs.

The compound N-(6-chloro-5-(2-methoxyphenoxy)[2,2′-bipyrimidinyl]-4-1-butyl benzenesulfonamide is isolated as its corresponding metal salt such as potassium, sodium, cesium, lithium, magnesium and zinc. Preferably the compound is isolated as its cesium salt.

We herein therefore disclose a novel cesium salt of N-(6-Chloro-5-(2-methoxyphenoxy)[2,2′-bipyrimidinyl]-4-t-butyl benzenesulfonamide.

In one of the preferred embodiment the invention discloses cesium salt of N-(6-chloro-5-(2-methoxyphenoxy)[2,2′-bipyrimidinyl]-4-t-butyl benzene sulfonamide in crystalline form, which is characterized by PXRD pattern with peaks at about 4.45, 9.02, 10.26, 12.94, 13.44, 14.04, 14.56, 15.22, 16.42, 16.68, 17.12, 18.01, 18.72, 19.50, 20.26, 20.61, 21.25, 21.63, 22.26, 22.49, 22.89, 23.22, 24.23, 25.15, 26.29, 26.82, 27.22, 27.48, 28.17 and 29.44°±0.2° (2θ) (FIG. 4).

(e) N-(6-chloro-5-(2-methoxyphenoxy)[2,2′-bipyrimidinyl]-4-t-butyl-benzene sulfonamide or its metal salts of formula (IV) is converted in to Bosentan using ethanediol in presence suitable base and optionally in presence of suitable phase transfer catalyst.

Suitable bases used in step-(e) is selected from alkali metals such as sodium, potassium, lithium; alkali hydrides such as sodium hydride lithium hydride, n-BuLi, LDA and KHMDS; alkali metal alkoxides such as sodium methoxide, sodium ethoxide, potassium t-butoxide, sodium t-butoxide; alkali hydroxides selected from sodium hydroxide, lithium hydroxide, potassium hydroxide and the like; suitable metal carbonates and bicarbonates selected from alkali and alkaline earth metal carbonates and bicarbonates, preferabally carbonates and bicarbonates of Na, K, Li, Ca, Mg, Cs and Zn; suitable organic bases selected from C₍₁₋₅₎ alkyl amines, C₍₁₋₅₎ substituted alkyl amines such as triethyl amine (TEA), diisopropyl amine, diisopropylethyl amine, heterocyclic saturated or unsaturated amines, preferably morpholine, piperidine, pyrollidine, Imidazole and pyridine; 2,4,6-collidine, 1,1,3,3,-tetramethyl guanidine. Suitable phase transfer catalyst which may be used are selected from tetra alkyl ammonium salts and crown ethers. Tetra alkyl ammonium salts may be selected from tetrabutyl ammonium iodide, tetrabutyl ammonium bromide and tetrabutyl ammonium chloride. Preferably, the crown ether used is selected from 18-crown-6.

In the present invention, the ethylene glycol is used as reactant as well as solvent. This improves the operational efficiency as well as the cost of production. Surprisingly, we found that, molar proportion of ethylene glycol (55 equi.) with respect to sulphonamide or its metal salts required for the conversion is also reduced.

Reaction is carried out at temperature 50-150° C., preferable 70-130° C., more preferably 80-120° C. and most preferably 90-110° C.

The Bosentan obtained has a purity of at least 92%, which is further purified by crystallization and recrystallization in suitable solvent.

The crystallization is carried out in suitable solvents such as alcohols, esters, chlorinated solvents like chloroform, dichloromethane, nitriles like acetonitrile, hydrocarbons like hexane, heptane, cyclohexane, toluene, xylene, chloro benzene, ketones like acetone, ethers like diethyl ether, 1,4-dioxane, DIPE, MTBE, THF and DMF, DMSO, DMA, formamide, NMP, 1,2-dimethoxy ethanol, 2-methoxy ethanol, 2-ethoxy ethanol, ethylene glycol, water or their suitable mixtures, and subsequently recrystallized from suitable solvents selected from any of the above.

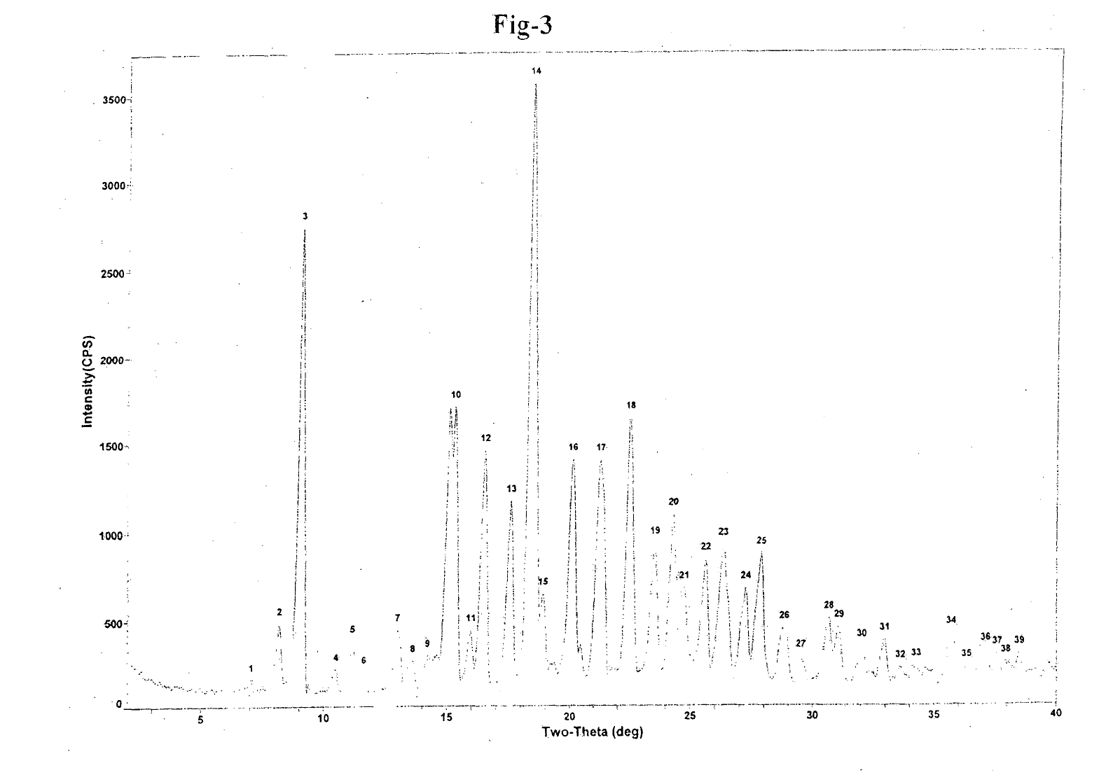

In one embodiment of the invention is disclosed new polymorphic form of Bosentan obtained according to the process of the present invention which is characterized by an XPRD pattern substantially in accordance with the pattern of FIG. 3. The polymorphic form of Bosentan obtained is also characterized by an XPRD peaks at about 7.03, 8.19, 9.13, 10.49, 11.18, 11.63, 13.00, 13.62, 14.22, 15.34, 15.98, 16.54, 17.60, 18.46, 18.90, 20.10, 21.26, 22.50, 23.54, 24.28, 24.73, 25.62, 26.32, 27.24, 27.86, 28.82, 29.49, 30.66, 31.07, 32.02, 32.95, 33.60, 34.26, 35.68, 36.32, 37.09, 37.57, 37.93 and 38.47°±0.2° (2θ) and has a melting point in the range of 128-130° C.

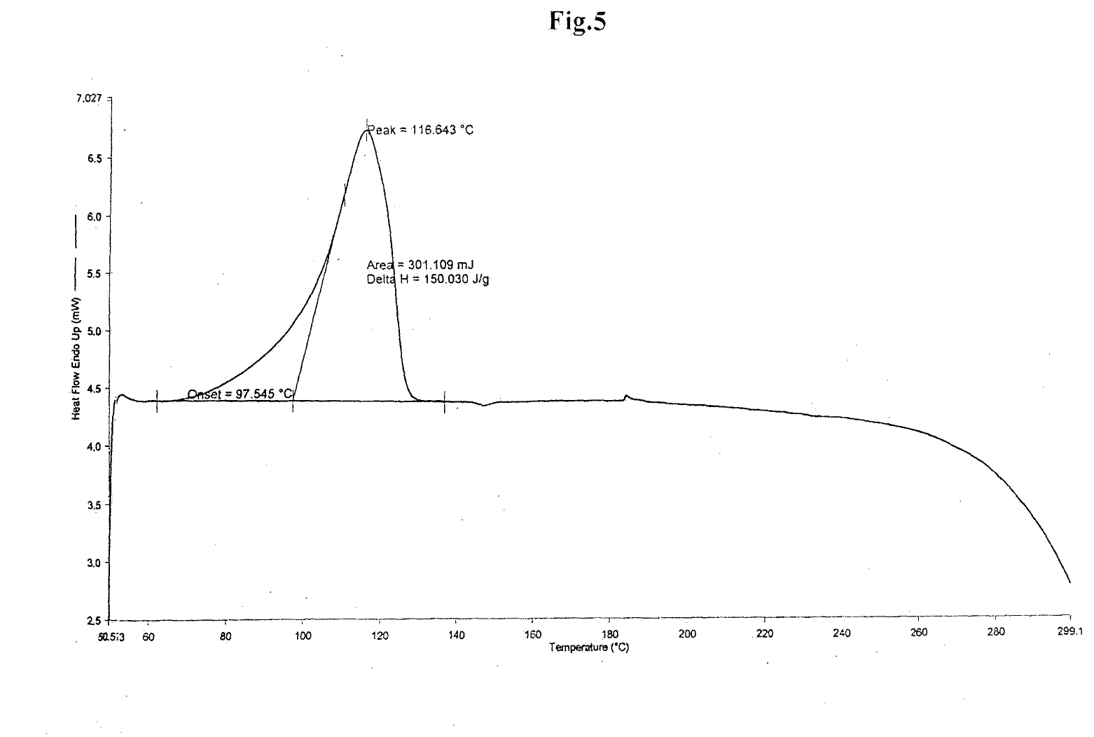

In a further aspect of the invention, the novel polymorphic form of Bosentan is characterized by a DSC endotherm at about 116° C. Preferably, the novel form of Bosentan has a DSC substantially as depicted in FIG. 5.

In one of the preferred embodiment the invention disclosed a process for the preparation of Bosentan using metal salts of N-(6-chloro-5-(2-methoxyphenoxy)[2,2′-bipyrimidinyl]-4-t-butyl-benzene sulfonamide reacted with ethanediol in presence suitable base and optionally in presence of suitable phase transfer catalyst.

In order to obtain marketing approval for a new drug product, manufacturers must submit to the regulatory authority evidence that the purity product is acceptable for administration to humans. Such a submission must include impurity profile of the product to demonstrate that the impurities are either absent, or present in a negligible amount. Different regulatory authorities have promulgated guidelines requiring applicants to identify the impurities present in the product and also disclose their concentration in the product. They also provide the maximum level of impurities allowable in the product. Thus, USFDA recommends that drug applicants identify all the impurities having concentration of 0.1% or greater than 0.1% in the active ingredient. Therefore, there is a need to check impurity profile and identify the impurities and also their concentration in the active ingredient.

The product mixture of a reaction rarely is a single compound pure enough to comply with pharmaceutical standards. Side products and byproducts of the reaction and adjunct reagents used in the reaction will, in most cases, be present. At certain stages during processing of the Bosentan contained in the product mixture into an active pharmaceutical ingredient, it must be analyzed for purity, typically by HPLC, LC-MS or GC analysis.

Generally, impurities (Side products, degradation product, byproducts and adjunct reagents) are identified spectroscopically and by other physical methods and then the impurities are associated with a peak position in a chromatogram. Thereafter, the impurity can be identified by its position in the chromatogram, which is conventionally measured in minutes between injection of the sample on the column and elution of the particular component through the detector, known as “retention time”. This time period varies daily based upon the condition of the instrumentation and many other factors. To mitigate the effect that such variations have upon accurate identification of an impurity, practitioners use “relative retention time” (RRT) to identified impurities. The RRT of an impurity is its retention time divided by the retention time of some reference marker. Thus, it is sometimes desirable to select an alternative compound that is added to, or is present in, the mixture in an amount significant enough to be detectable and sufficiently low as not to saturate the column and to use that as the reference marker.

Researchers and developers in drug manufacturing understand that a compound in a relatively pure state can be used as a reference standard” (a “reference marker is to similar to a reference standard but it is used for qualitative analysis) to quantify the amount of the compound in an unknown mixture. When the compound is used as an “external standard” a solution of a known concentration of the compound is analyzed by the same technique as the unknown mixture.

The reference standard compound also can be used to quantify the amount of another compound in mixture if the “response factor”, which compensates for differences in the sensitivity of the detector to the two compounds, has been predetermined.

The reference standard compound can even be used as an internal standard when the unknown mixture contains some of the reference standard compound by using a technique called “standard addition” wherein at least two samples are prepared by adding known and differing amounts of the internal standard. The proportion of detector response due to the reference standard compound that is originally in the mixture can be determined by extrapolation of a plot of detector response versus the amount of the reference standard compound that was added to each of the sample to zero.

In an embodiment is provided a novel compound of formula (III), as a reference standard for Bosentan.

In another aspect, the invention encompasses a process for synthesizing Compound of formula III by reacting N-(6-Chloro-5-(2-methoxyphenoxy)[2,2′-bipyrimidinyl]-4-t-butyl benzene sulfonamide with 4-t-Butyl benzene sulphonamide in a suitable solvent(s) and in presence of suitable base.

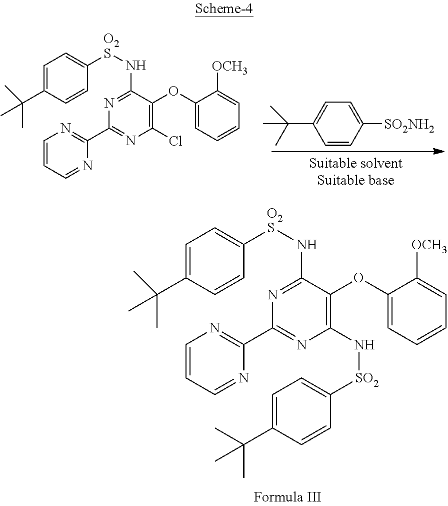

The organic solvents used in above reaction may be selected from alcohols like methanol, ethanol, isopropanol, butanol, 1,2-dimethoxy ethanol, 2-methoxy ethanol, 2-ethoxy ethanol, ethylene glycol and the like, esters like ethyl acetate, isopropyl acetate & the like, chlorinated solvents like chloroform, dichloromethane & the like, nitriles like acetonitrile & the like, hydrocarbons like toluene, xylene, chlorobenzene & the like, ketones like acetone & the like, ethers like diethyl ether, 1,4-dioxane, DIPE, MTBE, THF & the like, aprotic polar solvents such as DMF, DMSO, DMA & the like and suitable mixtures of one or more of the solvents described above. Suitable base used may be selected from hydroxides such as NaOH, KOH and like, carbonates such as NaHCO₃, Na₂CO₃, K₂CO₃ and like, hydrides such as NaH, n-BuLi, LDA and KHMDS and like or mixtures thereof.

The Bosentan of the present invention may be formulated into suitable pharmaceutical compositions by combining with a liquid or solid carrier, excipients etc. as is known in the art.

The invention is further described by the following examples, which are provided for illustration only and should not be construed to limit the scope of the invention.

### Example 1

### Preparation of 4,6-dihydroxy-5-(2-methoxy phenoxy) [2,2′]bipyrimidine

In a dry, 1 L round bottom flask 845 ml of methanol was taken. To this 16 g sodium metal was added at room temperature till it was dissolved, under cooling to obtain sodium methoxide. 98.23 g of diethyl-2-(2-methoxy phenoxy) malonate (0.34 mole) was added into it and stirred at room temperature for 30 min to 1 hrs and subsequently added 65 g pyrimidine-2-carboxamidine benzenesulfonate (0.23 moles) in one lot. Further the reaction mixture was stirred at room temperature for 15-30 min, then refluxed the reaction mixture for 4-5 hrs. The reaction mixture was cooled, dumped into cold water and adjusted to pH-2 using hydrochloric acid. The solid obtained was filtered off, washed with water till ‘neutral, suck dried to obtain 4,6-dihydroxy-5-(2-methoxy phenoxy)[2, 2’]bipyrimidine. Yield: 71.5 g, Purity: 99%.

% Water (KF):-10.11%

IR (KBr): (3256 cm⁻¹, 3128 cm⁻¹, 3080 cm⁻¹, 3014 cm⁻¹, 2507 cm⁻¹, 1747 cm⁻¹, 1606 cm⁻¹, 1568 cm⁻¹, 1504 cm⁻¹, 1454 cm⁻¹, 1429 cm⁻¹, 1402 cm⁻¹, 1323 cm⁻¹, 1168 cm⁻¹, 1149 cm⁻¹, 1115 cm⁻¹, 677 cm⁻¹, 634 cm⁻¹, 546 cm⁻¹.

NMR (300 MHz, DMSO D₆): δ=8.99 (d 2H), 7.02-6.99 (dd, 1H), 6.94-6.88 (m 1H), 6.80-6.74 (m, 1H), 6.90-6.65 (dd, 1H)

¹³C NMR (300 MHz, DMSO D₆) δ=55.786, 112.961, 113.336, 120.543, 121.387, 121.995, 122.905, 146.527, 148.475, 149.232, 156.742, 158.066, 159.993.

MS: m/z=312.8 [M⁺]

PXRD peaks at about 9.62, 11.50, 11.77, 12.40, 14.46, 14.81, 16.04, 19.40, 20.30, 21.16, 22.12, 23.22, 23.97, 24.72, 25.11, 25.72, 26.39, 27.13, 27.84, 28.50, 28.94, 29.40, 30.18, 30.68, 31.04, 32.16, 32.49, 33.10, 33.90, 34.33, 35.22, 35.76, 36.99, 37.60, 38.15 and 39.64°±0.2° (2θ) (FIG.-1).

Similarly, 4,6-dihydroxy-5-(2-methoxy phenoxy)[2,2′]bipyrimidine was prepared using different salts of pyrimidine-2-carboxamidine in different batches and the results are summarized in table 1 given below.

TABLE 1

| | Diethyl-2- | | | | | | | | | | |
|---|---|---|---|---|---|---|---|---|---|---|---|
| | (2- | | | | | | | | | % | |
| | methoxy | | pyrimidine-2- | | | Reaction | | | | water | |
| Example | phenoxy) | | carboxamidine | | | hrs/ | | Yield & | | by | % |
| No. | malonate | | salt | | Solvent | Temp. | | % Yield | | KF | Purity |
| Example | 1.5 | g | 1 | g | C₂H₅ONa | 3.5 | h | 0.595 | g | — | 98.36 |
| 2 | | | (benzene | | (in EtOH) | (80° C.) | | (53.38) | | | |
| | | | sulfonate) | | | | | | | | |
| Example | 62.81 | g | 27 | g | CH₃ONa | 3 | h | 37.4 | g | 7.34 | 99.39 |
| 3 | | | (acetate) | | (in MeOH) | (65° C.) | | (80.73) | | | |
| Example | 225 | g | 96.7 | g | CH₃ONa | 3 | h | 156 | g | 9.79 | 98.55 |
| 4 | | | (acetate) | | (in MeOH) | (65° C.) | | (94.02) | | | |
| Example | 66.6 | g | 18.72 | g | CH₃ONa | 3 | h | 24.5 | g | 0.5 | 98.51 |
| 5 | | | (HCl) | | (in MeOH) | (65° C.) | | (66.46) | | | |
| Example | 325.7 | g | 140 | g | CH₃ONa | 3 | h | 214 | g | 10.23 | 98.7 |
| 6 | | | (acetate) | | (in MeOH) | (65° C.) | | (89%) | | | |
| Example | 349 | g | 150 | g | CH₃ONa | 3 | h | 215 | g | 9.84 | 99.06 |
| 7 | | | (acetate) | | (in MeOH) | (65° C.) | | (83%) | | | |
| Example | 604 | g | 260 | g | CH₃ONa | 3 | h | 343 | g | 0.23 | 99.5 |

| | | | | |
|---|---|---|---|---|
| 8 | (acetate) | (in MeOH) | (65° C.) | (76.8%) |

### Example 9

### Preparation of 4,6-dihydroxy-5-(2-methoxy phenoxy) [2,2′]bipyrimidine

In a dry, 5 L round bottom flask 1.620 ml methanol was taken and 93.7 g sodium methoxide (1.73 mole), was added under cooling, and stirred till solid dissolved. 244.8 g diethyl-2-(2-methoxy phenoxy) malonate was added into it and stirred at room temperature for 30 min to 1 hrs and subsequently added 162 g pyrimidine-2-carboxamidine benzenesulfonate in one lot. The reaction mixture was stirred at room temperature for 15-30 min and the reaction mixture was refluxed for 4-5 hrs. The reaction mixture was cooled, dumped into cold water and adjusted to pH-2 using hydrochloric acid. The solid obtained was filtered and washed with water till neutral, suck dried to obtain 4,6-dihydroxy-5-(2-methoxy phenoxy)[2, 2′]bipyrimidine. Yield: 166.3 g Purity: 97.96%, % Water (KF):-9.13%.

1.2 g of above material was taken and dried in an oven at 100-120° C. for 6-7 hrs, cooled at room temperature and taken out. Yield: 0.949 g, HPLC purity: 99.05%.

% water by KF: 0.37%.

PXRD peaks at about 10.97, 11.42, 12.44, 14.74, 15.98, 17.00, 17.98, 18.89, 19.58, 20.56, 21.22, 22.48, 23.10, 23.96, 25.00, 25.64, 26.54, 27.22, 27.98, 28.96, 29.62, 30.22, 31.37, 32.88, 33.49, 34.56, 35.93, 36.64 and 37.01°±0.2° (2θ) (FIG.-2).

### Example 10

### Preparation of 4,6-dichloro-5-(2-methoxy phenoxy) [2,2]bipyrimidine

In a dry, 1 L round bottom flask, 70 g 4,6-dihydroxy-5-(2-methoxy phenoxy)[2,2′]bipyrimidine (0.22 mol) was taken. To this 90.73 g triethylamine (0.89 mole) was added and mixture was stirred, subsequently 137.4 g phosphorous oxychloride (0.89 mole) was added. Thick mass was obtained and subsequently the reaction mixture was stirred at 90-95° C. for 18-19 hrs and then the reaction mixture was dumped into ice cold water. The solid was filtered, washed with water, suck dried till constant weight. Yield: 61.5 g, Purity: 98.1%

Similarly, 4,6-dichloro-5-(2-methoxy phenoxy) [2,2′]bipyrimidine was prepared in different batches and the results are summarized in table 2 given below.

TABLE 2

| | Input | Output | % | % | Temp./reaction |
|---|---|---|---|---|---|
| Sr. No. | (g) | (g) | Purity | Yield | time |
| Example 11 | 22 | 19.2 | 92.62 | 78.05 | 90-95° C./22 hrs |
| Example 12 | 70 | 61.5 | 98.10 | 78.58 | 90-95° C./22 hrs |
| Example 13 | 58 | 60 | 98.84 | 92.51 | 90-95° C./22 hrs |
| Example 14 | 10 | 8.1 | 95.09 | 72.45 | 90-95° C./17 hrs |
| Example 15 | 70 | 62.8 | 97.33 | 80.24 | 90-95° C./18 hrs |
| Example 16 | 75 | 66.9 | 96.81 | 79.85 | 90-95° C./18 hrs |
| Example 17 | 155 | 136 | 92.45 | 78.24 | 90-95° C./18 hrs |
| Example 18 | 25 | 23 | 93.34 | 82.28 | 90-95° C./18 hrs |
| Example 19 | 50 | 46 | 97.2 | 82.28 | 90-95° C./18 hrs |
| Example 20 | 213 | 187 | 97.05 | 78.51 | 90-95° C./15 hrs |
| Example 21 | 212 | 195.8 | 96.42 | 82.6 | 90-95° C./16 hrs |

### Example 22

### Preparation of 4,6-dichloro-5-(2-methoxy phenoxy)[2,2′]bipyrimidine

In a dry, 1 L round bottom flask, 314.2 g phosphorous oxychloride (2.04 mole) was taken and cooled to 0 to 5° C. 208 g triethylamine (2.0 mole) was added into it through addition funnel. Subsequently added 16 g 4,6-dihydroxy-5-(2-methoxy phenoxy)[2,2′]bipyrimidine (0.51 mol) and stirred for 10-30 min. at same temperature. Then 11.2 mL water was added in 1 hour time intervals at 0 to 5° C. Thick mass was obtained, subsequently the reaction mixture was stirred at 90-95° C. for 18-20 hrs. The reaction mixture was dumped into ice cold water. Solid was obtained, which was filtered, washed with water, suck dried till constant weight. Yield: 165 g, Chemical purity: 97.03% (% Yield: 92.2%)

### Example 23

### Preparation of N-(6-Chloro-5-(2-methoxyphenoxy)[2,2′-bipyrimidinyl]-4-t-butyl benzenesulfonamide cesium salt

In a 3 L three neck flask, 142.9 g 4-tertbutylbenzene sulfonamide (0.67 mol) was taken, 218.3 g cesium carbonate and 1 L dimethyl formamide were added in to it and stirred for 10-30 min at room temperature. Subsequently 195 g 4,6-dichloro-5-(2-methoxy phenoxy) [2,2′]bipyrimidine (0.0.55 mol) was added and the reaction mixture was stirred for 18-20 hrs at 105-110° C. The reaction mixture was cooled, dumped into cold water and acidified with hydrochloric acid. Solid was obtained, which was filtered, washed with water, suck dried till constant weight. Yield: 341 g (92.8%), Chemical purity: 94.73%

This crude material was purified with isopropyl alcohol to give 304 g product with 97.8% purity.

10 g of above material was suspended in 150 mL water and stirred at 60-65° C. for 1-2 hr. The reaction mixture was cooled and filtered, suck dried till constant weight. Yield: 8 g. HPLC Purity: 98%

IR (KBr): 3506 cm⁻¹, 3433 cm⁻¹, 3063 cm⁻¹, 3036 cm⁻¹, 2958 cm⁻¹, 2904 cm¹, 2868 cm¹, 2843 cm⁻¹, 1643 cm⁻¹, 1591 cm⁻¹, 1552 cm⁻¹, 1494 cm⁻¹, 1448 cm⁻¹, 1431 cm⁻¹, 1392 cm⁻¹, 1305 cm⁻¹, 1280 cm⁻¹, 1247 cm⁻, 1220 cm⁻, 1207 cm⁻¹, 1178 cm⁻¹, 1138 cm⁻¹, 1080 cm⁻¹, 1041 cm⁻¹, 1012 cm⁻¹, 895 cm⁻¹, 844 cm⁻¹, 827 cm⁻¹, 792 cm⁻¹, 717 cm⁻¹, 655 cm⁻¹, 638 cm⁻¹, 584 cm⁻¹, 540 cm⁻¹.

¹H NMR (300 MHz, DMSO D₆): δ=8.98-8.97 (d, 2H), 7.89-7.87 (d, 2H), 7.60-7.58 (t, 1H), 7.28-7.26 (d, 2H), 7.08-7.06 (m, 1H), 6.98-6.94 (m, 1H), 6.80-6.76 (m, 1H), 6.42-6.39 (m, 1H), 3.82 (s, 3H), 1.21, (s, 9H).

¹³C NMR (300 MHz, DMSO D₆) δ=162.55, 159.63, 157.43, 156.97, 152.41, 148.79, 148.24, 145.85, 142.38, 132.98, 127.73, 123.87, 122.00, 121.16, 120.48, 113.17, 113.06, 99.52, 55.72, 34.39, 30.97.

MS: m/z=657.9 [M⁺Cs]

PXRD pattern with peaks at about 4.45, 9.02, 10.26, 12.94, 13.44, 14.04, 14.56, 15.22, 16.42, 16.68, 17.12, 18.01, 18.72, 19.50, 20.26, 20.61, 21.25, 21.63, 22.26, 22.49, 22.89, 23.22, 24.23, 25.15, 26.29, 26.82, 27.22, 27.48, 28.17 and 29.44°±0.2° (2θ)(FIG. 4).

Similarly, N-(6-Chloro-5-(2-methoxyphenoxy)[2,2′-bipyrimidinyl]-4-t-butyl benzene sulfonamide or its metal salt was prepared in different batches and the results are summarized in table 3 given below.

TABLE 3

| | | | | | | Sulfon- |
|---|---|---|---|---|---|---|
| | | | | | | amide/ |
| | | | | | | Sulfon- |
| | Input | Solvent/ | Output | % | % | amide |
| Sr. No | (g) | Catalyst | (g) | Purity | Yield | salt |
| Example 24 | 5 | DMF/CsCO₃ | 4.90 | 80.44 | 52 | Cs-salt |
| Example 25 | 40 | DMF/CsCO₃ | 50.9 | 97.96 | 67.5 | Cs-salt |
| Example 26 | 8 | DMF/CsCO₃ | 11.3 | 92.23 | 75.0 | Cs-salt |

### Example 27

### Preparation of 4-tert-butyl-N-[6-(2-hydroxyethoxy)-5-(2-methoxyphenoxy)-2-(2-pyrimidinyl)pyrimidin-4-yl]benzenesulfonamide

In a 25 mL one neck flask, 4.7 mL (0.083 moles) ethylene glycol was taken and to it was added 0.92 g (0.0023 moles) sodium hydroxide and stirred till solid dissolved. Subsequently 1 g chloro sulfonamide derivatives (0.0015 moles) and 0.12 g 18-Crown-6 were added. The reaction mixture was stirred at 80-85° C. for 11-12 hr. The reaction mixture was cooled, dumped into cold water and acidified with hydrochloric acid. Solid was obtained, filtered, washed with water, suck dried till constant weight it. Yield 0.721 g (86%), HPLC Purity: 94.65%.

Purification of Bosentan obtained above is carried out in a similar way as disclosed in Example-28, to obtain pure Bosentan.

### Example 28

### Preparation of 4-tert-butyl-N-[6-(2-hydroxyethoxy)-5-(2-methoxyphenoxy)-2-(2-pyrimidinyl)pyrimidin-4-yl]benzenesulfonamide

In a 2 L 4 neck flask 795 mL ethylene glycol was taken and to it was added 7.4 g sodium metal portion wise. Stirred till total sodium metal was dissolved. Subsequently added 70 g chloro sulfonamide derivatives obtained above, and the reaction mixture was stirred at 100° C. for 27-30 hr. The reaction mixture was cooled and dumped into ice cold water. Filtered the solid and washed with water till neutral, suck dried till constant weight. Yield: 72.5 g Purity: 95.66%

The crude Bosentan (10 g) was further stirred with a mixture of methanol and isopropyl acetate (1.1) at reflux temperature, till solid dissolves. It was cooled at 0-2° C. and filtered.

Yield: 7.6 g, Purity: 97.8%, which on further crystallization, from a mixture of ethanol and water gave Bosentan with 99.13% purity.

Similarly, 4-tert-butyl-N-[6-(2-hydroxyethoxy)-5-(2-methoxyphenoxy)-2-(2-pyrimidinyl)pyrimidin-4-yl]benzene sulfonamide was prepared by using alkali metal as catalyst and using N-(6-Chloro-5-(2-methoxyphenoxy)[2,2′-bipyrimidinyl]-4-t-butyl benzenesulfonamide or its metal salt in different batches and the results are summarized in table 4 given below.

TABLE 4

| | In- | | Out- | | | Reaction | Sulfonamide/ |
|---|---|---|---|---|---|---|---|
| | put | Cata- | put | % | % | hrs/ | Sulfonamide |
| Sr. No | (g) | lyst | (g) | Purity | Yield | Temp. | salt |
| Example 29 | 70 | Na/ | 72.3 | 94.66 | 98.5 | 100-5° C./ | Sulfonamide |
| | | EG | | | | 21 hr | |

Purification of Bosentan obtained above is carried out in a similar way as disclosed in Example-28, to obtain pure Bosentan.

### Example 30

### Preparation of 4-tert-butyl-N-[6-(2-hydroxyethoxy)-5-(2-methoxyphenoxy)-2-(2-pyrimidinyl)pyrimidin-4-yl]benzenesulfonamide

In a 50 mL 2 necked flask 11.3 mL ethylene glycol was taken. To this 512 mg potassium-tert-butoxide was added in one lot. The reaction mixture was stirred till it dissolves. Subsequently 1 g chloro sulfonamide derivative obtained above was added. (0.0019 moles) and further the reaction mixture was stirred at 100-105° C. for 28-30 hr. The reaction mixture was cooled and dumped into ice cold water and acidified with hydrochloric acid. Filtered the solid and washed with water till neutral, suck dried till constant weight Yield: 850 mg, (% yield: 81%), Chemical Purity: 69.78%

Purification of Bosentan obtained above is carried out in a similar way as disclosed in Example-28, to obtain pure Bosentan.

Similarly, 4-tert-butyl-N-[6-(2-hydroxyethoxy)-5-(2-methoxyphenoxy)-2-(2-pyrimidinyl)pyrimidin-4-yl]benzene sulfonamide was prepared by using alkali metal alkoxide as catalyst and using N-(6-Chloro-5-(2-methoxyphenoxy)[2,2′-bipyrimidinyl]—4-1-butyl benzenesulfonamide or its metal salt in different batches and the results are summarized in table 5 given below.

TABLE 5

| | In- | | Out- | | | | Sulfonamide/ |
|---|---|---|---|---|---|---|---|
| | put | | put | % | % | Reaction | Sulfonamide |
| Sr. No | (g) | Catalyst | (g) | Purity | Yield | hrs/Temp. | salt |
| Example | 9.5 | CH₃ONa/ | 9.6 | 93.22 | 96.36 | 100-5° C./ | Sulfonamide |
| 31 | | EG | | | | 24 hr | |
| Example | 1 | C₂H₅ONa/ | 0.930 | 93.77 | 88.68 | 100-5° C./ | Sulfonamide |
| 32 | | EG | | | | 22 hr | |

Purification of Bosentan obtained above is carried out in a similar way as disclosed in Example-28, to obtain pure Bosentan.

### Example 33

### Preparation of 4-tert-butyl-N-[6-(2-hydroxyethoxy)-5-(2-methoxyphenoxy)-2-(2-pyrimidinyl)pyrimidin-4-yl]benzenesulfonamide

In a 250 mL 3 neck flask, 129.7 mL ethylene glycol was taken. 2.27 g sodium hydroxide (0.056 mole) was added, in one lot. Reaction mixture was heated at 55-60° C. and stirred to dissolve. Subsequently, 25 g N-(6-Chloro-5-(2-methoxyphenoxy)[2,2′-bipyrimidinyl]-4-t-butyl benzenesulfonamide cesium salt was added into it and the reaction mixture was stirred at 100-105° C. for 5-6 hr. The reaction mixture was cooled and dumped into ice cold water and acidified. Filtered the solid and washed with water till neutral, suck dried and dried further till constant weight. Yield: 18.9 g, (% yield: 90.1%) Chemical Purity: 92.14%

Purification of Bosentan obtained above is carried out in a similar way as disclosed in Example-28, to obtain pure Bosentan.

PXRD peaks at about 7.03, 8.19, 9.13, 10.49, 11.18, 11.63, 13.00, 13.62, 14.22, 15.34, 15.98, 16.54, 17.60, 18.46, 18.90, 20.10, 21.26, 22.50, 23.54, 24.28, 24.73, 25.62, 26.32, 27.24, 27.86, 28.82, 29.49, 30.66, 31.07, 32.02, 32.95, 33.60, 34.26, 35.68, 36.32, 37.09, 37.57, 37.93 and 38.47°±0.2° (2θ) (FIG.-3).

M.P.-120-122° C.

% of water by KF: 3.27%

Similarly, 4-tert-butyl-N-[6-(2-hydroxyethoxy)-5-(2-methoxyphenoxy)-2-(2-pyrimidinyl)pyrimidin-4-yl]benzenesulfonamide was prepared by using alkali metal hydroxide as catalyst and using N-(6-Chloro-5-(2-methoxyphenoxy)[2,2′-bipyrimidinyl]-4-t-butyl benzene sulfonamide or its metal salt in different batches and the results are summarized in table 6 given below.

TABLE 6

| | | | | | | | Sulfonamide/ |
|---|---|---|---|---|---|---|---|
| | Input | | Output | % | % | | Sulfonamide |
| Sr. No | (g) | Catalyst | (g) | Purity | Yield | Temp./Reaction hrs. | salt |
| Example 34 | 3 | NaOH/EG | 2.44 | 94.11 | 97 | 100-5° C./5 hr | Cs-salt |
| Example 35 | 25 | NaOH/EG | 18.90 | 92.14 | 90.17 | 100-5° C./4.5 hr | Cs-salt |
| Example 36 | 100 | NaOH/EG | 76 | 91.99 | 90.64 | 100-5° C./5 hr | Cs-salt |
| Example 37 | 0.5 | LiOH/EG | 0.330 | 92.69 | 78.71 | 100-5° C./4.5 hr | Cs-salt |
| Example 38 | 5 | Na salt EG | 5.1 | 95.9 | 97.26 | 100-5° C./4.5 hr | Sulfonamide |
| Example 39 | 5 | NaOH | 5 | 95.3 | 95.3 | 100-5° C./5.5 hr | Sulfonamide |
| Example 40 | 25 | NaOH | 18.4 | 92.08 | 87.7 | 100-5° C. | Cs-salt |
| Example 41 | 5 | NaOH | 2.7 | 89.9 | 65.0 | 80-5° C./46 hr | Cs-salt |
| Example 42 | 2 | NaOH | 1.2 | 93.4 | 73.12 | 90-5° C./60 hr | Cs-salt |
| Example 43 | 1 | KOH | 0.702 | 27.3 | 83.7 | 100-5° C./7 hr | Cs-salt |
| Example 44 | 1 | NaOH | 0.760 | 87.6 | 90.6 | 90-5° C./50 hr | Cs-salt |
| Example 45 | 1 | Mono Na | 0.849 | 92.69 | — | 100-5° C./24 hr | Sulfonamide |
| | | salt/EG | | | | | |

Purification of Bosentan obtained above is carried out in a similar way as disclosed in Example-28, to obtain pure Bosentan.

### Example 46

### Preparation of 4-tert-butyl-N-[6-(2-hydroxyethoxy)-5-(2-methoxyphenoxy)-2-(2-pyrimidinyl)pyrimidin-4-yl]benzenesulfonamide

In a 25 mL one neck flask, 3 mL (0.054 moles) ethylene glycol was taken and into it 403 mg (0.0038 moles) sodium carbonate and 18-crown-6 (PTC) as a catalyst was added and stirred for 30 mins at 60-65° C. Subsequently chlorosulfonamide cesium salt 500 mg were added and the reaction mass was heated at 100-105° C. for 24 hrs. The reaction mixture was cooled, dumped into cold water and acidified with hydrochloric acid. Solid obtained was filtered, washed with water, suck dried till constant weight. HPLC Purity: 89%.

Purification of Bosentan obtained above is carried out in a similar way as disclosed in Example-28, to obtain pure Bosentan.

### Example 47

### Preparation of 4-tert-butyl-N-[6-(2-hydroxyethoxy)-5-(2-methoxyphenoxy)-2-(2-pyrimidinyl)pyrimidin-4-yl]benzenesulfonamide

In a 50 mL one neck flask, 3 mL ethylene glycol was taken and into it 805 mg sodium carbonate and 100 mg tetrabutyl ammonium iodide as catalyst was added and stirred for 30 mins at 60-65° C. Subsequently chlorosulfonamide cesium salt 500 mg was added and the reaction mass was heated at 100-105° C. for 28 hrs. The reaction mixture was cooled, dumped into cold water and acidified with hydrochloric acid. Solid obtained was filtered, washed with water, suck dried till constant weight. Crude yield: 307 mg (73%); HPLC purity 93.5%.

Purification of Bosentan obtained above is carried out in a similar way as disclosed in Example-28, to obtain pure Bosentan.

### Example 48

### Preparation of 4-tert-butyl-N-[6-(2-hydroxyethoxy)-5-(2-methoxyphenoxy)-2-(2-pyrimidinyl)pyrimidin-4-yl]benzenesulfonamide

In a 500 mL one neck flask, 91 mL ethylene glycol was taken and into it 12.8 gm sodium carbonate and 1.5 g DMAP(PTC) as catalyst was added and stirred for 3 hrs at 60-65° C. Subsequently chlorosulfonamide cesium salt 10 g in 70 ml THF and stirred the reaction mixture at reflux temperature for 135 hrs. After the completion of reaction solvent was distilled off. The reaction mass was cooled, dumped into cold water and acidified with hydrochloric acid. Solid obtained was filtered, washed with water, suck dried till constant weight. Crude yield 8.5 g (99% yield); HPLC purity 93.54%.

Purification of Bosentan obtained above is carried out in a similar way as disclosed in Example-28, to obtain pure Bosentan.

### Example 49

### Preparation of 4-tert-butyl-N-[6-(2-hydroxyethoxy)-5-(2-methoxyphenoxy)-2-(2-pyrimidinyl)pyrimidin-4-yl]benzenesulfonamide

In a 50 mL one neck flask, 3 mL ethylene glycol was taken and into 110 mg sodium t-butoxide and 25 mg tetrabutyl phosphonium bromide was added and stirred at 60-65° C. till the clear solution. Subsequently chlorosulfonamide cesium salt 0.5 g was added and stirred the reaction mixture at 90-95° C. for 98 hrs. After the completion of reaction solvent was distilled off. The reaction mass was cooled, dumped into cold water and acidified with hydrochloric acid. Solid obtained was filtered, washed with water, suck dried till constant weight. Crude yield 165 mg (40%) HPLC purity 95.99%.

Purification of Bosentan obtained above is carried out in a similar way as disclosed in Example-28, to obtain pure Bosentan.

### Example 50

### Preparation of N,N′-(5-(2-methoxyphenoxy)-2,2′-bipyrimidine-4,6-diyl)bis(4-tert-butylbenzenesulfonamide

In 250 mL 3 neck round bottom flask, 6.5 g N-(6-Chloro-5-(2-methoxyphenoxy)[2,2′-bipyrimidinyl]-4-t-butyl benzene sulfonamide, 3.9 g 4-t-Butyl benzene sulphonamide and 65 mL DMF was taken at room temperature. The reaction mixture was heated at 145-150° C. 1.87 g anhydrous K₂CO₃ was added to the reaction mixture and heated at 145-150° C. for 16 hours. Further 2.69 g 4-t-butyl benzene sulphonamide and 1.19 g anhydrous K₂CO₃ was added and further reaction mixture was heated at 145-150° C. for 24 hrs. The reaction mixture was cooled to room temperature and dumped into water. Yellow solid was precipitated out which was stirred for 15 min. The reaction mixture was acidified with 50% HCl till pH 3 and stirred for 15 min. Sticky solid was formed which was isolated with ethyl acetate. Free flowing yellow material was obtained. Solid was filtered and washed with water and then washed with ethyl acetate. Yield:-2.5 g, HPLC: 95.6%, unwanted material and only 1% N,N′-(5-(2-methoxyphenoxy)-2,2′-bipyrimidine-4,6-diyl)bis(4-tert-butylbenzenesulfonamide).

Filtrate was combined and ethyl acetate layer was separated out. Ethyl acetate layer was washed with water, followed by brine, dried over anhydrous sulfate and solvent was removed under vacuum to give 7.4 g, N,N′-(5-(2-methoxyphenoxy)-2,2′-bipyrimidine-4,6-diyl)bis(4-tert-butylbenzenesulfonamide), HPLC Purity=11.14%.

(6 g) of N,N′-(5-(2-methoxyphenoxy)-2,2′-bipyrimidine-4,6-diyl)-bis(4-tert-butylbenzenesulfonamide) was taken in 50 mL conical flask and 40 mL isopropyl alcohol was added and stirred for 5 min., when a suspension was formed. The reaction mixture was heated at 80° C. for 5 min. to obtain clear solution. The solution was allowed to stand at room temperature for 2 hrs and yellow solid was precipitated. Solid product was filtered and washed with 10 ml isopropyl alcohol and dried. Solid was obtained (700 mg), which contains 3.65% N,N-(5-(2-methoxyphenoxy)-2,2′-bipyrimidine-4,6-diyl)bis(4-tert-butylbenzenesulfonamide). Filtrate was concentrated to give 4.8 g N,N′-(5-(2-methoxyphenoxy)-2,2′-bipyrimidine-4,6-diyl)bis(4-tert-butylbenzenesulfonamide). This was further purified with methanol to give 950 mg N,N′-(5-(2-methoxyphenoxy)-2,2′-bipyrimidine-4,6-diyl)bis(4-tert-butylbenzenesulfonamide).

(LC/MS result):

| | RT | % Area | |
|---|---|---|---|
| | 21.008 | 30.4613 | |
| Bis sulphonamide impurity | 21.524 | 36.7419 | =RT = 24.304, % |
| Area = 30.61% | 24.304 | 30.6152 | |

We claim:

1. A process for the preparation of Bosentan comprising,
(a) reacting a salt of the pyrimidine-2-carboxamidine of formula (V) with 2-(2-methoxyphenoxy) malonic acid diethyl ester of formula (VIII) in presence of suitable alkali metal alkoxide in suitable solvent to give compound of formula (II). 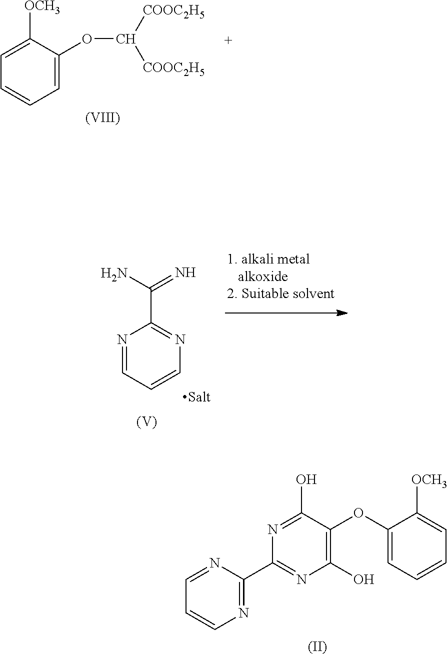
(b) converting the compound of formula (II) into 4,6-dichloro-5-(2-methoxy phenoxy)-[2,2′]bipyrimidine of formula (III) using suitable dehydrohalogenating agent in presence of suitable base. 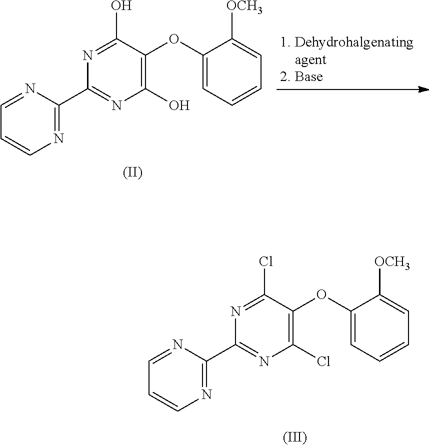
(c) reacting pyrimidine dihalide compound of the formula (III) with 4-tert-butyl-benzene sulfonamide in presence of suitable base in a suitable solvent to obtain N-(6-chloro-5-(2-methoxyphenoxy)[2,2′-bipyrimidinyl]-4-t-butyl-benzene sulfonamide of the formula (IV). 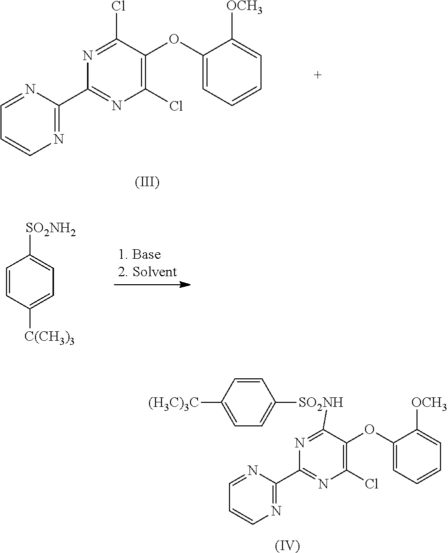
(d) converting the N-(6-chloro-5-(2-methoxyphenoxy)[2,2′-bipyrimidinyl]-4-t-butyl benzene sulfonamide or its metal salts of formula (IV) in to Bosentan by treating with ethanediol in presence of suitable base and optionally in presence of suitable phase transfer catalyst.

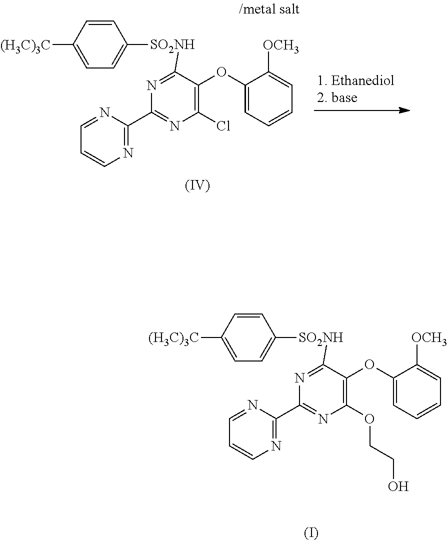

2. The process as claimed in claim 1 wherein in step (a) the salt of the pyrimidine-2-carboxamidine is selected from hydrochloride, hydrobromide, acetate, sulfate and benzene sulfonate salts.

3. The process as claimed in claim 1 wherein in step (b), the dehydrohalogenating agent is selected from phosphorous oxychloride, phosphorous pentachloride, phosphorous trichloride, oxalyl chloride, pyrophosphorous oxychloride or their suitable mixture and the base is selected from suitable tertiary amines.

4. The process as claimed in claim 3, wherein the tertiary amines are selected from such as triethyl amine, trimethyl amine, triisopropyl amine and diisopropyl ethylamine.

5. The process as claimed in claim 1 wherein in step (c), said suitable base is slected from alkali carbonates, alkali hydroxides and alkali earth metal carbonates.

6. The process as claimed in claim 1 wherein step (c), said suitable solvent is selected from toluene, cyclohexane, dimethyl formamide, dimethyl sulfoxide, dimethyl acetamide or mixtures thereof.

7. The process as claimed in claim 1 wherein in step (d), the suitable base is selected from alkali metals, alkali metal hydrides, alkali metal alkoxides and alkali hydroxides.

8. The process as claimed in claim 1 wherein in step (d), wherein the suitable phase transfer catalyst is selected from suitable Crown ethers or quaternary salts selected from R₄N⁺X⁻, R₄P⁺X⁻ and R₄As⁺X⁻, wherein R represents an alkyl group and X represents suitable halogen atom.

9. An intermediate of structural formula-(II):

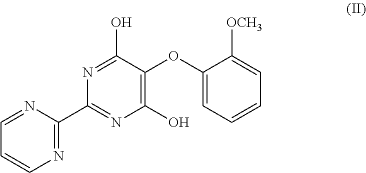

10. The compound of formula (II) as claimed in claim 9, characterized by NMR (300 MHz, CDCl₃) δ (ppm) values of 8.99 (d 2H), 7.02-6.99 (dd, 1H), 6.94-6.88 (m 1H), 6.80-6.74 (m, 1H), 6.90-6.65 (dd, 1H).

11. The compound of formula (II) as claimed in claim 9, further characterized by a PXRD pattern with peaks at about 9.62, 11.50, 11.77, 12.40, 14.46, 14.81, 16.04, 19.40, 20.30, 21.16, 22.12, 23.22, 23.97, 24.72, 25.11, 25.72, 26.39, 27.13, 27.84, 28.50, 28.94, 29.40, 30.18, 30.68, 31.04, 32.16, 32.49, 33.10, 33.90, 34.33, 35.22, 35.76, 36.99, 37.60, 38.15 and 39.64°±0.2° (2θ).

12. The compound of formula (II) as claimed in claim 9-11, further characterized by a PXRD pattern substantially as depicted in FIG. 1.

13. A new polymorphic form of compound of formula (II) as claimed in claim 9, characterized by a PXRD pattern with peaks at about 10.97, 11.42, 12.44, 14.74, 15.98, 17.00, 17.98, 18.89, 19.58, 20.56, 21.22, 22.48, 23.10, 23.96, 25.00, 25.64, 26.54, 27.22, 27.98, 28.96, 29.62, 30.22, 31.37, 32.88, 33.49, 34.56, 35.93, 36.64 and 37.01±0.2° (2θ).

14. The polymorphic form of compound of formula (II) as claimed in claim 13, further characterized by a PXRD pattern as depicted in FIG. 2.

15. The compound of formula (II) as claimed in claim 9-14, containing from about 0.1-10% water by weight.

16. The compounds of formula (II) as claimed in claims 9-15 which are suitable as intermediate for the preparation of Bosentan.

17. N-(6-Chloro-5-(2-methoxyphenoxy)[2,2′-bipyrimidinyl]-4-t-butyl benzene sulfonamide cesium salt.

18. The compound as claimed in claim 17, further characterized by a PXRD pattern with peaks at about 4.45, 9.02, 10.26, 12.94, 13.44, 14.04, 14.56, 15.22, 16.42, 16.68, 17.12, 18.01, 18.72, 19.50, 20.26, 20.61, 21.25, 21.63, 22.26, 22.49, 22.89, 23.22, 24.23, 25.15, 26.29, 26.82, 27.22, 27.48, 28.17 and 29.44°±0.2° (2θ).

19. The compound as claimed in claim 17, further characterized by a PXRD pattern as depicted in FIG. 4.

20. The compound of claims 17-19, which are suitable for the preparation of Bosentan.

21. A compound of formula-(III):

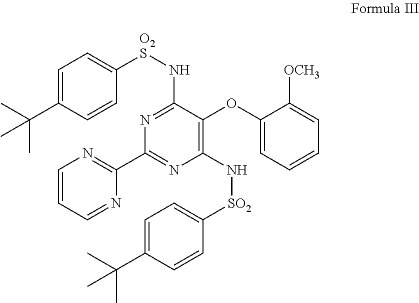

22. The compound of claim 21, which is an impurity of Bosentan.

23. The impurity of claim 21 obtained in the process of preparing Bosentan according to the present invention.

24. A process for preparing compound of formula (III) comprising reacting N-(6-Chloro-5-(2-methoxyphenoxy)[2,2′-bipyrimidinyl]-4-t-butyl benzene sulfonamide and its salts with 4-t-Butyl benzene sulphonamide in a suitable solvent and in presence of suitable base.

25. The process as claimed in claim 24, wherein suitable solvent is selected from alcohols, esters, chlorinated solvents, nitriles, hydrocarbons, ketones, ethers, aprotic polar solvents or their suitable mixtures.

26. The process as claimed in claim 24, wherein said suitable base used is selected from metal hydroxides, metal carbonates, metal hydrides or mixtures thereof.

27. A process of purification of Bosentan prepared as claimed in claim 1, which comprises crystallization and recrystallization from suitable solvent selected from alcohols, esters, chlorinated solvents, nitriles, ketones, ethers, DMF, DMSO, DMA, formamide, NMP, 1,2-dimethoxy ethanol, 2-methoxy ethanol, 2-ethoxy ethanol, ethylene glycol, water or their suitable mixtures.

28. The process as claimed in claim 27, wherein Bosentan obtained is having at least 99% purity by HPLC.

29. Bosentan prepared according to process of claim 1, containing from about 2-4% water by weight.

30. A pharmaceutical composition comprising Bosentan together with a liquid or solid carrier, and suitable excipients, wherein the Bosentan is a product of any one of the preceding claims.
